(19) United States
(12) Patent Application Publication
(10) Pub. No.: US 2009/0143788 A1
(43) Pub. Date: Jun. 4, 2009

(54) Navigation method and system for drilling operation in spinal surgery

(75) Inventors: Jin-Jing Fang, Tainan (TW); Ruey-Mo Lin, Tainan (TW); Yu-Hung Yen, Taichung County (TW)

Correspondence Address: BACON & THOMAS, PLLC, 625 SLATERS LANE, FOURTH FLOOR, ALEXANDRIA, VA, 22314-1176, US

(73) Assignee: National Cheng Kung University, Tainan City (TW)

(21) Appl. No.: 12/230,202

(22) Filed: Aug. 26, 2008

(30) Foreign Application Priority Data
Dec. 4, 2007 (TW) 096146103

(51) Int. Cl. G06K 9/00; A61B 19/00

(52) U.S. Cl. 606/130; 382/132

(57) ABSTRACT

The invention is to provide a navigating method and system for drilling operation in spinal surgery. The method and system is applied to the subjects who receive pedicle insertion operation in spinal surgery. The specific vertebras are under radiography using X-ray machine at both anterior/posterior and lateral views. These imaging are then transmitted into a computer system for image distortion correction and registration. By drawing the safety regions of the pedicle on the indicated vertebra in both AP and Lat. images, three-dimensional safety volumes of the pedicle of the vertebra are developed by intersection operation. Therefore, prior to and during the clinical spine operation, surgeons are able to safe navigation by referring the referential orientation via the developed system.

### BACKGROUND OF THE INVENTION

1. Field of the Invention

The present invention relates to a navigation method and system for a drilling operation in spinal surgery.

2. Description of the Prior Art

Scientific and technological breakthroughs have constantly been driving diverse livings toward great abundance; on the other hand, it leads to a variety of modern diseases of civilization nevertheless, which indeed bother our minds a great deal. The pathologies of congenital and acquired scoliosis or spur and the like happened without being carefully noticed are subject to the frequent negligence in watching out one's own posture and the balance of physical strength for the busy life, and the deficiency to pay attention to the importance of the physical and mental relaxation seriously. Especially for the contemporaries of nowadays who make use of modern technological products (for instance: to sustain in clicking a keyboard by the right hand for long); hence, an annoyance of suffering a sore waist and an aching back happens at the beginning, it means their spines are already subject to some kinds of pressures. Without a timely therapeutic advice or a medical treatment, the scoliosis or the spur and so on will creep gradually into the patients and annoy them greatly. Up to such an extent, the sufferers have no choice but look for an orthopedist for a spinal correction therapy. A serious deformity of the spine demands an invasive vertebra drilling operation for open spinal surgery, where transpedicular screws are inserted for an internal fixation to carry out spinal correction. The radical part of the spinal correction surgery is performing a drilling operation at the selected spot (the pedicle) of a patient spine, where the drilling must be accurate and effective enough to proceed into the next move. Surgeons quite often face a spine mutilated beyond recognition when performing the drilling operation; in addition, only a tiny area on the pedicle that allows the insertion of the transpedicular screws (shown in FIG. 1), and meshed central nerves overspread its vicinity. Any incautious drilling of an imprecise angle will no doubt hurt nearby nerves, which probably brings about paraplegia to the patient. Therefore, the risk of this surgery is a high and demands an extreme caution. A multiple years of training is a must to accumulate enough experience of comfortable space sense and hand feel to smoothly accomplish the surgery. Accordingly, a provision of a navigation method and system for a drilling operation in the spinal surgery is offered to the orthopedists (especially for those who are just taking up office) for the assistance in checking the dissimilarities of simulated trajectory prior to the actual surgery. The advantage of the provision could be multifold, which features: to smooth the actual surgery, to abstain patients from suffering excess pains, to help orthopedists in accumulating the experience of comfortable space sense and hand feel promptly for the pedicle drilling, and to help orthopedists in promptly boosting their experience in the spinal surgery.

### SUMMARY OF THE INVENTION

An objective of the present invention is to provide a navigation method and system for a drilling operation in spinal surgery, which assists to guide surgeons to operate the drilling operation of the spinal surgery smoothly, and to keep patients from suffering excess pains.

Another object of the present invention is to provide a navigation method and system for a drilling operation in spinal surgery to orthopedists, who are executing the surgical procedure, for the assistance in accumulating the experience of the spinal surgery.

The present invention is a navigation method for a drilling operation in spinal surgery, which comprises the following steps:

A patient who is undergoing a surgical procedure lies prone on an operating table; To acquire an anterior/posterior (A/P) digital X-ray image and a lateral (Lat) digital X-ray image for the selected vertebra of the patient;

To perform image distortion correction and registration for the A/P and Lat. images;

To plan and draw a two-dimensional safety region for the pedicle of the selected vertebra on the A/P and Lat images that already underwent image distortion correction and registration;

To adjust the two-dimensional safety region to shrink into an effective two-dimensional safety region according to the diameter of the drill head for the actual drilling;

To obtain a three-dimensional safety reconstruction for the pedicle on the selected vertebra by executing a projective intersection operation for the two-dimensional safety region, followed by an operation of inclination adjustment; and

Spinal surgeons perform a drilling probe against the selected vertebra to gauge a passage through the three-dimensional safety reconstruction.

In the aforesaid navigation method for a drilling operation in spinal surgery, the drawing of the two-dimensional safety region comprises the edit on the A/P image, for the two-dimensional safety region for the left pedicle of the selected vertebra on an A/P image, and for the two-dimensional safety region for the right pedicle of the selected vertebra on an A/P image, and the edit on the Lat image, for the two-dimensional safety region of the selected vertebra on an Lat image.

In the aforesaid navigation method for a drilling operation in spinal surgery, the three-dimensional safety reconstruction for the selected vertebra comprises a three-dimensional safety volume of the left pedicle, which is the result of the projective intersection operation by the effective two-dimensional safety region of the left pedicle on an A/P image and the effective two-dimensional safety region on a Lat image, and a three-dimensional safety volume of the right pedicle, which is the result of the projective intersection operation by the effective two-dimensional safety region of the right pedicle on an A/P image and the effective two-dimensional safety region on a Lat image.

In the aforesaid navigation method for a drilling operation in spinal surgery, the operation of inclination adjustment of the three-dimensional safety reconstruction is implemented by establishing an exit face and an enter face of the two-dimensional safety region on the A/P image of the left pedicle and the A/P image of the right pedicle respectively, during planning the two-dimensional safety region, and followed by computing the horizontal distance between the enter face and the exit face; next, establish a two-dimensional safety region of the pedicle in profile on the Lat Image, where the shape is like a parallelogram, and compute the maximum horizontal distance of the parallelogram as the height of the safety region of the pedicle. The inclinations of the left and the right pedicles are then calculated to adjust the inclinations for the three-dimensional safety volume of the left and the right pedicles.

A further object of the present invention is to provide a navigation system for a drilling operation in spinal surgery, which is a navigation system for performing the drilling operation of the spinal surgery on the selected vertebra for patients lying prone on the operating table, comprising: a drilling probe capable of being traceable, offering a surgeon the probe setting and orientation for the selected vertebrae; and a computer, undergoing the following steps:

To acquire an anterior/posterior (A/P) digital X-ray image and a lateral (Lat) digital X-ray image for the selected vertebra of the patient;

To perform image distortion correction and registration for the A/P and Lat images;

To receive a two-dimensional safety region of the selected vertebra that was planned and drawn on the A/P and Lat images that already underwent the image distortion correction and registration;

To adjust the two-dimensional safety region to shrink into an effective two-dimensional safety region according to the diameter of the drill head for the actual drilling;

To obtain a three-dimensional safety reconstruction for the selected vertebra by executing a projective intersection operation for the two-dimensional safety region, followed by an operation of inclination adjustment; and

To receive the three-dimensional safety reconstruction of the selected vertebra that is under a comparison with a drilling probe through which the drilling procedure is then safely guided to pass the three-dimensional safety reconstruction.

In the aforesaid navigation system for a drilling operation in spinal surgery, the computer is able to offer the edit on the A/P image, for the two-dimensional safety region for the left pedicle of the selected vertebra on an A/P image, and for the two-dimensional safety region for the right pedicle of the selected vertebra on an A/P image, and the edit on the Lat image for the two-dimensional safety region of the selected vertebra on an Lat image.

In the aforesaid navigation system for a drilling operation in spinal surgery, the three-dimensional safety reconstruction for the selected vertebra comprises a three-dimensional safety volume of the left pedicle, which is the result of the projective intersection operation by the effective two-dimensional safety region of the left pedicle on an A/P image and the effective two-dimensional safety region on a Lat image, and a three-dimensional safety volume of the right pedicle, which is the result of the projective intersection operation by the effective two-dimensional safety region of the right pedicle on an A/P image and the effective two-dimensional safety region on a Lat image.

In the aforesaid navigation system for a drilling operation in spinal surgery, the operation of inclination adjustment of the three-dimensional safety reconstruction is implemented by establishing an exit face and an enter face of the two-dimensional safety region on the A/P image of the left pedicle and the A/P image of the right pedicle respectively, during planning the two-dimensional safety region, and followed by computing the horizontal distance between the enter face and the exit face; next, establish a two-dimensional safety region of the pedicle in profile on the Lat Image, where the shape is like a parallelogram, and compute the maximum horizontal distance of the parallelogram as the height of the safety region of the pedicle. The inclinations of the left and the right pedicles are then calculated to adjust the inclinations for the three-dimensional safety volume of the left and the right pedicles.

A still further object of the present invention is the readable means used to realize a navigation system for a drilling operation in spinal surgery, which stores a series of executable steps of a program code as follows:

To acquire an anterior/posterior (A/P) digital X-ray image and a lateral (Lat.) digital X-ray image for the selected vertebra of a patient;

To perform image distortion correction and registration for the digital X-ray images;

To receive a two-dimensional safety region for the selected vertebra that was planned and drawn on the A/P and Lat images that already underwent the image distortion correction and registration;

To adjust the two-dimensional safety region to shrink into an effective two-dimensional safety region according to the diameter of the drill head for the actual drilling;

To obtain a three-dimensional safety reconstruction for the selected vertebra by executing a projective intersection operation for the two-dimensional safety region, followed by an operation of inclination adjustment; and

To receive the three-dimensional safety reconstruction of the selected vertebra that is under a comparison with a drilling probe through which the drilling procedure is then safely guided to pass the three-dimensional safety reconstruction.

In the aforesaid computer readable means which is realized as a navigation system for a drilling operation in spinal surgery, which comprises the offering for the edit on the A/P image, for the two-dimensional safety region for the left pedicle of the selected vertebra on an A/P image, and for the two-dimensional safety region for the right pedicle of the selected vertebra on an A/P image, and the edit on the Lat image for the two-dimensional safety region of the selected vertebra on an Lat image.

In the aforesaid computer readable means which is realized as a navigation system for a drilling operation in spinal surgery, the three-dimensional safety reconstruction for the selected vertebra comprises a three-dimensional safety volume of the left pedicle, which is the result of the projective intersection operation by the effective two-dimensional safety region of the left pedicle on an A/P image and the effective two-dimensional safety region on a Lat image, and a three-dimensional safety volume of the right pedicle, which is the result of the projective intersection operation by the effective two-dimensional safety region of the right pedicle on an A/P image and the effective two-dimensional safety region on a Lat image.

In the aforesaid computer readable means which is realized as a navigation system for a drilling operation in spinal surgery, the operation of inclination adjustment of the three-dimensional safety reconstruction is implemented by establishing an exit face and an enter face of the two-dimensional safety region on the A/P image of the left pedicle and the A/P image of the right pedicle respectively during planning the two-dimensional safety region, and followed by computing the horizontal distance between the enter face and the exit face; next, establish a two-dimensional safety region of the pedicle in profile on the Lat image, where the shape is like a parallelogram, and compute the maximum horizontal distance of the parallelogram as the height of the safety region of the pedicle. The inclinations of the left and the right pedicles are then calculated to adjust the inclinations for the three-dimensional safety volume of the left and the right pedicles.

### BRIEF DESCRIPTION OF THE DRAWINGS

FIG. 1 is a schematic diagram depicting a drilling operation on the selected pedicle of the vertebra; and

FIG. 2 is a block flow chart showing the exemplified embodiment of the present invention.

### APPENDIX

Reference PIC. 1 depicts the images of pedicles of vertebras C4, T8, T12, and L4

Reference PIC. 2 depicts the images of the lumbar vertebrae of a human spine

Reference PIC. 3 depicts the images of the thoracic vertebrae of a human spine

Reference PIC. 4 depicts the images of a planned two-dimensional safety region

Reference PIC. 5 depicts the images of a drawn two-dimensional safety region

Reference PIC. 6 depicts the images of an adjusted two-dimensional safety region

Reference PIC. 7 depicts the images of a preliminarily reconstructed three-dimensional safety volume

Reference PIC. 8 depicts the images of a drawn safety region for a decided inclination

Reference PIC. 9 is a schematic drawing depicting the correction of the inclination

Reference PIC. 10 depicts the images of three-dimensional safety volumes of the left and right pedicles for a corrected inclination

### DETAILED DESCRIPTION OF THE INVENTION

To achieve the foregoing objective of the present invention, the techniques adopted and the functions achieved are detailed described with reference to the following preferred embodiments and the accompanying drawings, which helps to arrive at a thorough comprehension.

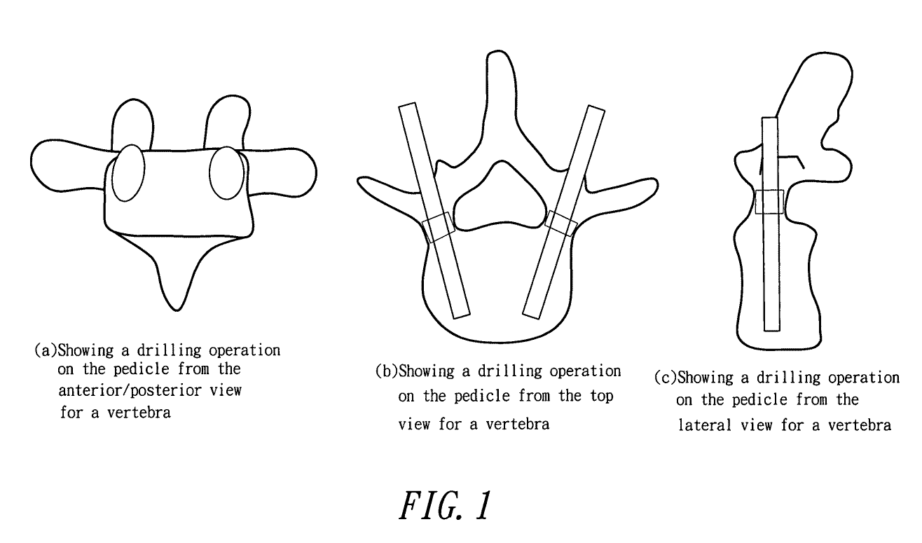

The exemplified embodiment of the present invention is a navigation method and system used in a bone drilling operation for spinal surgery, which targets to guide spinal surgeons through the performing of the surgical procedure for a patient, assisting to smooth the surgery through to accomplishment. In general, this surgical procedure demands the patient to lie prone on an operating table, and to take radiographs by an X-ray machine, where the radiographs are required to take from different angles for a selected vertebrae for the actual surgery, and the surgeon, for executing the surgery, observes the fluoroscopic images to decide the spot and angle for the drilling by means of his/her pertinent experience; at this moment, the odds actually are dependent on the expertise of the surgeons. The present invention is conceived by drawing a three-dimensional safety workspace of the drilling operation on the radiographs based on the visual judgment criterion of surgeons, which helps to guide the surgeon through the trajectory during the drilling, to accomplish the surgery smoothly and to accumulate the experience of space sense and hand feel. To achieve this objective and capacity, the present invention plans the two-dimensional safety region of the drilling trajectory for the pedicle based on the definition of the pedicle by anatomy and the location of the pedicle on the radiographs. Then, it follows the procedure by drawing the two-dimensional safety region on both the anterior/posterior (A/P) image and the lateral (Lat) image, and by establishing a three-dimensional safety workspace by a projective intersection operation, which is used to guide the spinal surgeons. During the pedicle drilling for a patient, shown in FIG. 1; the actual drilling of a specific depth is subject to the locations and angles of the left and right pedicles.

An example of the vertebras C4, T8, T12, and L4 from a donated Gross is shown in Reference PIC. 1. The (a) column and (c) column are the real pictures of the vertebras. To tie both ends of each vertebra above by iron wires followed by taking pictures, the X-ray images of A/P orientation are in (b) column and the X-ray images of Lat orientation are in (d) column. The images in the (b) column of the Reference PIC. 1 apparently exhibit the location of the two windings of iron wire on each pedicle, which learns that the projections of the enter face and the exit face of the pedicle on A/P surface are not completely overlapping, where C4 and T8 vertebras have less overlapping for their two windings each, because human's pedicles actually have an inclined angle, resulting in the more deflected for the enter face than the exit face toward the outer side of the vertebra; (d) column shows the locations of the imaging of the enter face and the exit face for the pedicle of each vertebra on the Lat. images, which apparently shows that the vertebras T8, T12 and L4 have their enter face and exit face separated for each pedicle on the Lat images, while the vertebra C4 fails to separate the enter face from the exit face for its pedicle on both AP and Lat orientations according to the geometrical structure of the pedicle in anatomy; therefore, the method of planning three-dimensional safety workspace of the present invention doesn't apply to the cervical vertebrae, only apply to thoracic vertebrae and lumbar vertebrae.

Reference PIC. 2 & 3 are the images of safety regions for the lumbar vertebrae and the thoracic vertebrae respectively. In the Reference PIC. 2 & 3, the winding on the A/P image and the interior of the quadrilateral formed by four points on the Lat image are the projections of the pedicle on the X-ray images, where the hole to be drilled and the screw to be inserted are done within this pedicle portion. Suppose the trajectory of the drilling pierces to the outside of the pedicle, major blood vessels could be damaged to bleed aplenty, whereas piercing through the interior of the pedicle, nerves could be destroyed to cause badly harm. Therefore, surgeons mark a definite location of the pedicle on the X-ray images as the safety region for the drilling operation prior to the actual surgery. The present invention proceeds to a later planning and processing based on this designated safety region. Once a vertebra of a patient is selected for undergoing digital X-ray photograph, the images are subject to distortion correction, which is extracting imaging from the calibration board during the extraction of imaging from the X-ray machine (including a few images by the calibration board for a known angle). Since X-ray is penetrable into objects, metallic balls (for instance: steel balls) or other substance capable of absorbing X-ray are used for the calibration points for the calibration board, where the X-ray images of the calibration board show the required calibration points during the image distortion correction. After the operations of image processing and camera calibration techniques, two calibrated images are obtained.

It is then to plan a safe region for drilling on the digital radiographs A/P and Lat, which is a two-dimensional safety region. Each vertebra has a pair pedicles located at the left and the right, the left pedicle and the right pedicle; therefore, the projection regions for the left pedicle and the right pedicle can be planned on the A/P images by means of digital X-ray photography, and a lateral projection region for the pedicle can also be planned on the Lat image. Referring to Reference PIC. 4, the computer of the present invention has up to six sub-windows for its human interface, where the sub-windows on top row display two X-ray A/P images and one Lat image from left to right in full view, the sub-windows on the bottom row display the enlarged images for the square frames of their top-row counterparts. Since the three square frames on the top row must have their selected vertebra to be enlarged to be the same one; therefore, a correction for each square frame to have same y value for the x-y plane images, to fix the three enlarged regions with the same position along the y axis, and the enlarged images on the sub-windows of the bottom row are the A/P and Lat images for the same vertebra.

The two-dimensional safety region for the selected vertebra is then drawn on the A/P and Lat images, which is done by using the mouse to select a few coordinates through clockwise or counterclockwise within the planned two-dimensional safety region on the three enlarged images shown in Reference PIC. 4 Follow these orders of selection; a closed area is formed as in Reference PIC. 5, where six coordinates are selected on the A/P images to draw a closed hexagon, while four coordinates on the Lat image to form a parallelogram. The purpose of planning and drawing a two-dimensional safety region is to establish a three-dimensional safety workspace; therefore, it is essential to consider that the drill head used in the spinal surgery is a cylinder with a specific radius. Hence, the edited and drawn two-dimensional safety region demands an adjustment of shrinking, where the diameter value of the drill head is required to enter by the operator, which results in a shrinking for the edge of the original two-dimensional safety region by an amount of a radius, and forms an effective two-dimensional safety region, shown in Reference PIC. 6, wherein the inner frame is the boundary for the adjusted effective two-dimensional safety region.

By following the aforesaid approach, the effective two-dimensional safety region for the left pedicle on A/P (shown in Reference PIC. 6 left bottom figure), the effective two-dimensional safety region for the right pedicle on A/P (shown in Reference PIC. 6 middle bottom figure), and the effective two-dimensional safety region on Lat (shown in Reference PIC. 6 right bottom figure) for the selected vertebra are established. After the execution of the projective intersection operation, the three-dimensional safety workspace of the left pedicle for the selected vertebra (obtained from taking the effective two-dimensional safety region for the left pedicle A/P and the effective two-dimensional safety region for the Lat through the projective intersection operation), and the three-dimensional safety workspace of the right pedicle for the selected vertebra (obtained from taking the effective two-dimensional safety region for the right pedicle A/P and the effective two-dimensional safety region for the Lat through the projective intersection operation). The preliminary constructions of the three-dimensional safety workspaces for the left and right pedicles are shown in Reference PIC. 7.

The foregoing two three-dimensional safety workspaces, obtained by taking the projective intersection operation for the three effective two-dimensional safety regions, are a bit different from the pedicle volume obtained by the anatomy. As the patient is lying prone, the pedicle volume of the thoracic and lumbar vertebras by the anatomy is not perpendicular to the bed, but a pillar-shaped space a bit inclined to the spine interior. In general, human pedicles are inclined about 20 degrees to 30 degrees, and the present invention will make an adjustment based on this fact to the foregoing three-dimensional safety workspace, to make the result more closer to the one from the anatomy.

The adjusted angle for the inclination is determined in the course of planning the two-dimensional safety region, which adopts the steps as that of drawing the two-dimensional safety region, selecting six points to establish a red closed hexagon (shown in Reference PIC. 8(a)), where the hexagon is the area for the drill head to leave safely from the pedicle, which is taken as two-dimensional safety region exit face Lₒᵤₜ for the left pedicle. Once this is done, the system program will draw a green hexagon of the same size (shown in Reference PIC. 8(b)), where the hexagon is the area for the drill head to enter safely into the pedicle, which is taken as two-dimensional safety region enter face Lᵢₙ for the left pedicle. The procedure follows by moving and adjusting the horizontal position of the Lᵢₙ by operating the mouse, and to ascertain the selection of an adequate place for the Lᵢₙ by clicking the left button of the mouse, and the program is ready to compute the horizontal distance D_{left} between the Lᵢₙ and the Lₒᵤₜ, followed by the same steps to draw the two-dimensional safety region exit face Rₒᵤₜ for the left pedicle (shown in Reference PIC. 8(c)), the enter face Rᵢₙ (shown in Reference PIC. 8(d)), and the horizontal distance D_{right} between the Rᵢₙ and the Rₒᵤₜ. The lateral projection of the pedicle is then drawn on the Lat image, which has a shape like a parallelogram (shown in Reference PIC. 8(e)), and the height H of the safety space of the pedicle is the maximum horizontal distance of the quadrilateral formed by the P₁P₂P₃P₄ of the image. The program will then compute the inclined angle of the left pedicle Angle_{left} through D_{left} and H, and compute the inclined angle of the right pedicle Angle_{right} through D_{right} and H (shown in Reference PIC. 9). After the computation has done for the inclined angles for the left and right pedicles, the program proceeds to make an inclination correction for the three-dimensional safety workspaces for the left and right pedicles, to accomplish the three-dimensional safety workspaces with inclination corrected (shown in Reference PIC. 10), which approaches the workspace defined by the anatomy much closer.

Once the three-dimensional safety workspace for the selected vertebra is obtained with the inclination corrected, spine surgeons are ready for the comparison with the drilling trajectory prior to the actual invasive drilling operation of the spinal surgery, where the comparison means to replace the real drill head by a drilling probe. When the surgeon performs a simulation by operating the drilling probe for the selected vertebra, the orientation of the probe is designed to be traceable for detection (for instance: by an optical tracking device, a mechanical measuring machine, a magnetica tracking device and the like), for the comparison with the established three-dimensional safety workspace. Once the orientation of the drilling probe fits the three-dimensional safety workspace, the actual drilling is commencing; otherwise, a modification is required for the location or the angle until a fit for the three-dimensional safety workspace is arrived, and it is then ready for performing the drilling. Through this navigation approach, the safety of the surgical operation and the odds of success are tremendously boosted.

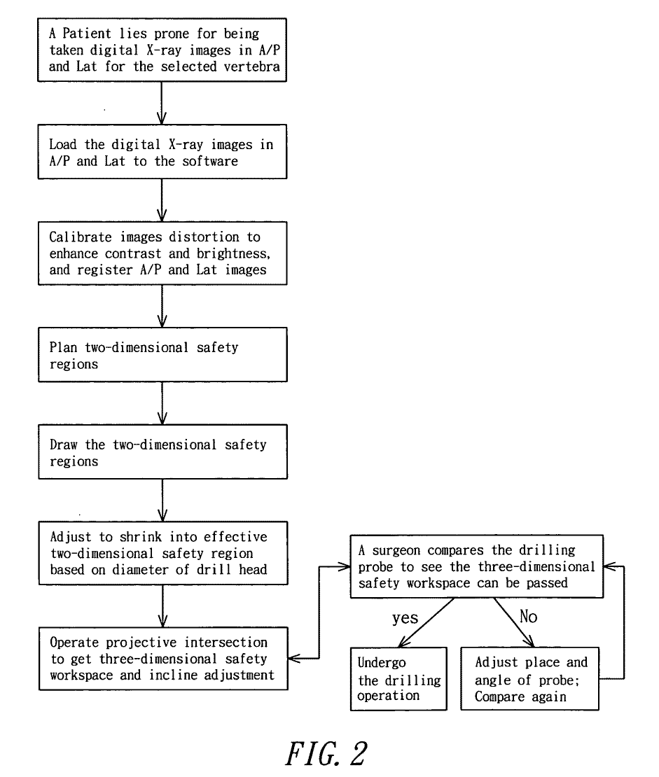

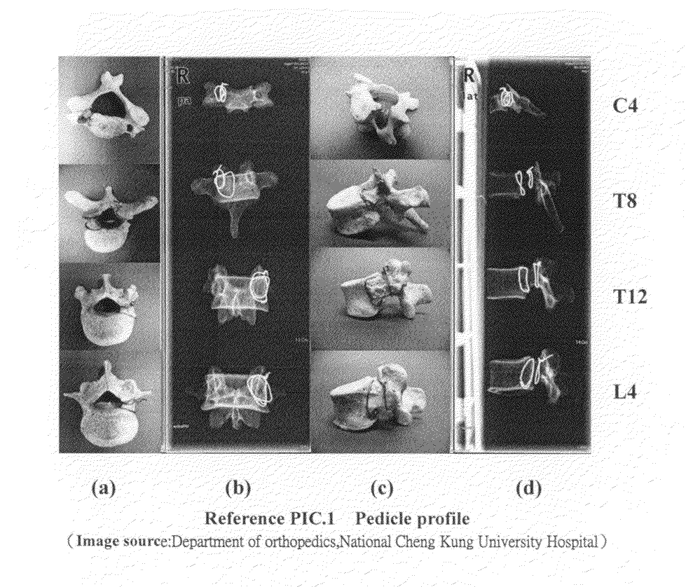

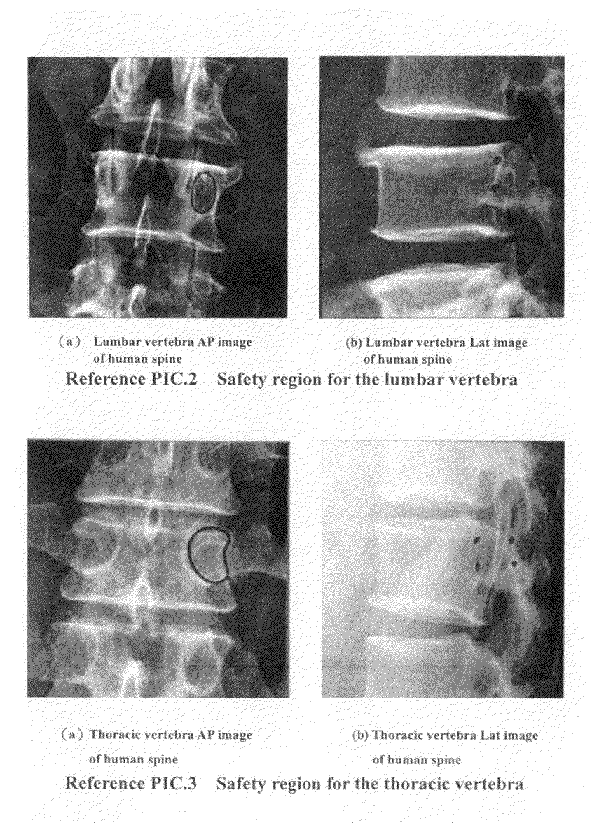

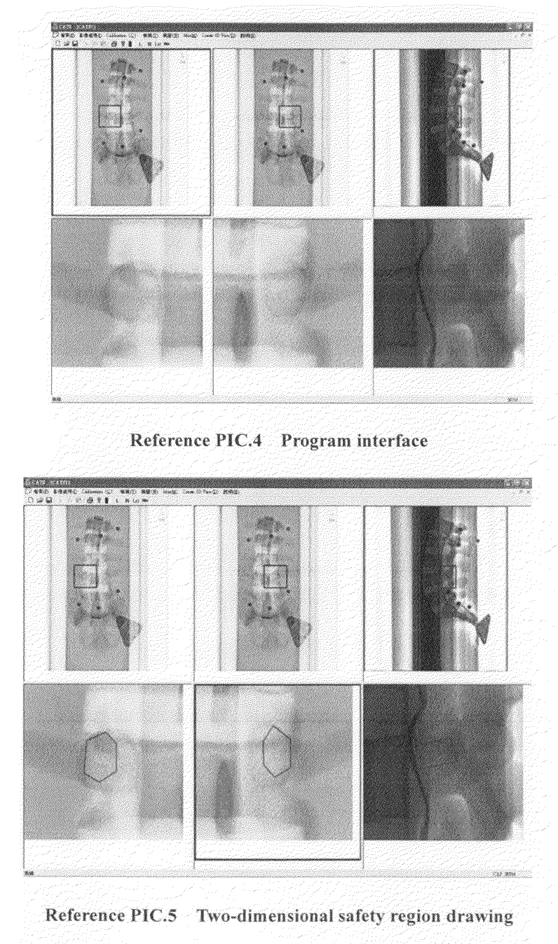

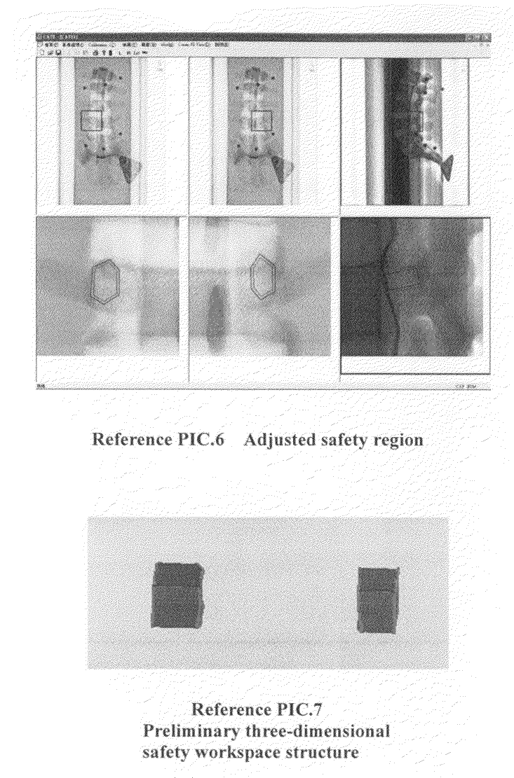

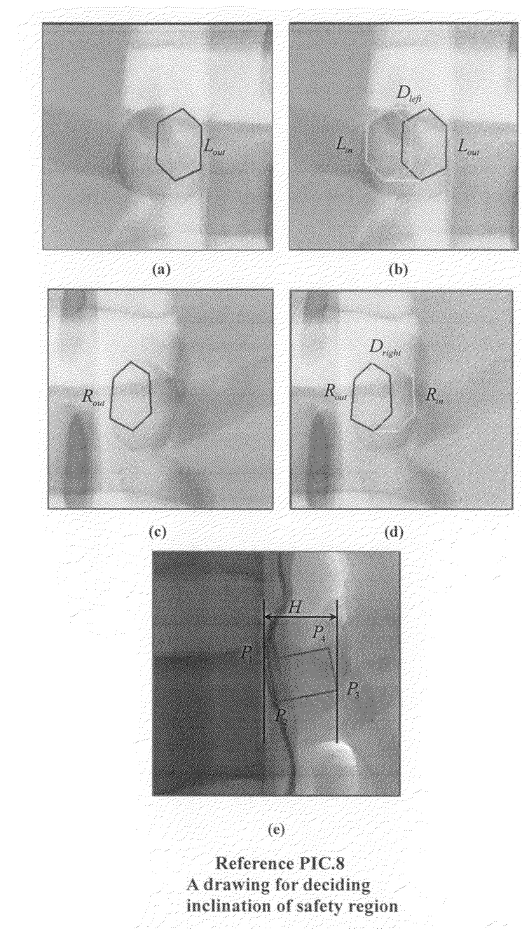

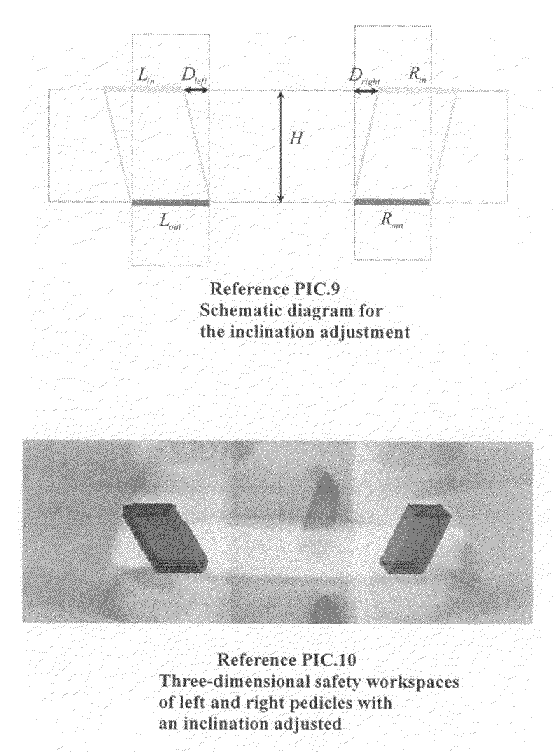

Accordingly, the exemplified embodiment of the present invention of a navigation method and system for a drilling operation in spinal surgery, comprises a drilling probe to be traceable for detection and a computer, and performs the detailed steps as follows (referring to FIG. 2):
(I.) Firstly, to acquire A/P and Lat images for the selected vertebra from the patient lying in prone on the bed and these digital radiographs are stored in the computer, where the computer contains a readable means, for storing the program code for executing the following steps; then, to carry out image distortion correction and registration; and to perform image processing for the original A/P and Lat images, for the enhancement of the contrast and brightness of the images, and to perform the registration for the A/P and Lat. images.
(II.) After the registration operation manipulated on the A/P and Lat images, a two-dimensional safety region is planned on an enlarged image of the above, which has a two-dimensional safety region for the left pedicle A/P, a two-dimensional safety region for the right pedicle A/P, and a two-dimensional safety region for Lat; followed by drawing the three two-dimensional safety regions, and adjusting to shrink into the effective two-dimensional safety regions based on the radius of the drill head.
(III.) To take the effective two-dimensional safety region for the left pedicle A/P, or the effective two-dimensional safety region for the right pedicle A/P, separately with the effective two-dimensional safety region for Lat in executing projective intersection operation to obtain a three-dimensional safety workspace, followed by an operation of inclination correction.
(IV.) A spine surgeon simulates a drilling orientation by operating a drilling probe against the patient body, where the location of the probe is designed to be traceable for the detection and stored in the computer for the calculation, which helps to know in advance that the probe orientation fits the three-dimensional safety workspace, and the actual drilling is kicking off; otherwise, a modification is undergone for the position and angle to be ready for another trial.

Since the present invention is a navigation method and system used for a drilling operation for the spinal surgery, where the objective can be achieved by executing the program code stored in a computer readable means (for instance: floppy disk, DVD, CD and the like). The present invention can be realized by loading the stored program to a computer followed by the execution of the program.

To summarize the foregoing description, the present invention is a navigation method used in a drilling operation of a spinal surgery and a system to be realized by the method. The program code for executing the steps of the navigation method stored in a readable means of the computer system is a brand new invention, which is fully complied with the novelty, creativeness and usefulness requirements of patentability, may obtain a patent thereof.

The above disclosed subject matter is merely a preferred exemplified embodiment of the present invention, which is not intended for limiting the scope of the claims of the present invention. Any equivalent variations fall within the technological thoughts of the present invention are therefore intended to be embraced by the present invention.

What is claimed is:

1. A navigation method used for a drilling operation for the spinal surgery, comprising the following steps:
A patient who is undergoing a surgical procedure lying prone on an operating table;
to acquire an anterior/posterior (AP) digital X-ray image and a lateral (Lat) digital X-ray image for the selected vertebra of the patient;
to perform image distortion correction and registration for the AP and Lat images;
to plan and draw a two-dimensional safety region for the pedicle of the selected vertebra on the AP and Lat images that already undergoing image distortion correction and registration;
to adjust the two-dimensional safety region to shrink into an effective two-dimensional safety region according to the diameter of the drill head for the actual drilling;
to obtain a three-dimensional safety reconstruction for the pedicle on the selected vertebra by executing a projective intersection operation for the two-dimensional safety region, followed by an operation of inclination adjustment; and
spinal surgeons performing a drilling probe against the selected vertebra to gauge a passage through the three-dimensional safety reconstruction.

2. A navigation method used for a drilling operation for the spinal surgery as in claim 1 wherein the drawing of the two-dimensional safety region comprises the edit on the AP image, for the two-dimensional safety region for the left pedicle of the selected vertebra on an AP image, and for the two-dimensional safety region for the right pedicle of the selected vertebra on an AP image, and the edit on the Lat image for the two-dimensional safety region of the selected vertebra on an Lat image.

3. A navigation method used for a drilling operation for the spinal surgery as in claim 2 wherein the three-dimensional safety reconstruction for the selected vertebra comprises a three-dimensional safety volume of the left pedicle, which is the result of the projective intersection operation by the effective two-dimensional safety region of the left pedicle on an AP image and the effective two-dimensional safety region on a Lat image, and a three-dimensional safety volume of the right pedicle, which is the result of the projective intersection operation by the effective two-dimensional safety region of the right pedicle on an AP image and the effective two-dimensional safety region on a Lat image.

4. A navigation method used for a drilling operation for the spinal surgery as in claim 1 wherein the operation of inclination adjustment of the three-dimensional safety reconstruction is implemented by establishing an exit face and an enter face of the two-dimensional safety region on the AP image of the left pedicle and the AP image of the right pedicle respectively, during planning the two-dimensional safety region, and followed by computing the horizontal distance between the enter face and the exit face; next, establishing a two-dimensional safety region of the pedicle in profile on the Lat image, where the shape is like a parallelogram, and computing the maximum horizontal distance of the parallelogram as the height of the safety region of the pedicle, and the inclinations of the left and the right pedicles being then calculated to adjust the inclinations for the three-dimensional safety volume of the left and the right pedicles.

5. A navigation system used for a drilling operation in the spinal surgery, for performing the drilling operation of the spinal surgery on the selected vertebra for patients lying prone on the operating table, comprising:
a drilling probe capable of being traceable, offering a surgeon the probe
setting and orientation for the selected vertebrae; and
a computer, undergoing the following steps:
to acquire an anterior/posterior (AP) digital X-ray image and a lateral (Lat) digital X-ray image for the selected vertebra of the patient;
to perform image distortion correction and registration for the AP and Lat images;
to receive a two-dimensional safety region of the selected vertebra that was planned and drawn on the AP and Lat images that already undergoing the image distortion correction and registration;
to adjust the two-dimensional safety region to shrink into an effective two-dimensional safety region according to the diameter of the drill head for the actual drilling;
to obtain a three-dimensional safety reconstruction for the selected vertebra by executing a projective intersection operation for the two-dimensional safety region, followed by an operation of inclination adjustment; and
to receive the three-dimensional safety reconstruction of the selected vertebra that is under a comparison with a drilling probe through which the drilling procedure being then safely guided to pass the three-dimensional safety reconstruction.

6. A navigation system used for a drilling operation for the spinal surgery as in claim 5 wherein the computer is able to offer the edit on the AP image, for the two-dimensional safety region for the left pedicle of the selected vertebra on an AP image, and for the two-dimensional safety region for the right pedicle of the selected vertebra on an AP image, and the edit on the Lat image for the two-dimensional safety region of the selected vertebra on an Lat image.

7. A navigation system used for a drilling operation for the spinal surgery as in claim 6 wherein the three-dimensional safety reconstruction for the selected vertebra comprises a three-dimensional safety volume of the left pedicle, which is the result of the projective intersection operation by the effective two-dimensional safety region of the left pedicle on an AP image and the effective two-dimensional safety region on a Lat image, and a three-dimensional safety volume of the right pedicle, which is the result of the projective intersection operation by the effective two-dimensional safety region of the right pedicle on an AP image and the effective two-dimensional safety region on a Lat image.

8. A navigation system used for a drilling operation for the spinal surgery as in claim 5 wherein the operation of inclination adjustment of the three-dimensional safety reconstruction is implemented by establishing an exit face and an enter face of the two-dimensional safety region on the AP image of the left pedicle and the AP image of the right pedicle respectively, during planning the two-dimensional safety region, and followed by computing the horizontal distance between the enter face and the exit face; next, establishing a two-dimensional safety region of the pedicle in profile on the Lat image, where the shape is like a parallelogram, and computing the maximum horizontal distance of the parallelogram as the height of the safety region of the pedicle, and the inclinations of the left and the right pedicles being then calculated to adjust the inclinations for the three-dimensional safety volume of the left and the right pedicles.

9. A readable means used in a navigation system for a drilling operation in spinal surgery, which stores a program for executing the following steps by a computer:
to acquire an anterior/posterior (AP) digital X-ray image and a lateral (Lat.) digital X-ray image for the selected vertebra of a patient;
to perform image distortion correction and registration for the digital X-ray images;
to receive a two-dimensional safety region for the selected vertebra that was planned and drawn on the AP and Lat images that already underwent the image distortion correction and registration;
to adjust the two-dimensional safety region to shrink into an effective two-dimensional safety region according to the diameter of the drill head for the actual drilling;
to obtain a three-dimensional safety reconstruction for the selected vertebra by executing a projective intersection operation for the two-dimensional safety region, followed by an operation of inclination adjustment; and
to receive the three-dimensional safety reconstruction of the selected vertebra that being under a comparison with a drilling probe through which the drilling procedure being then safely guided to pass the three-dimensional safety reconstruction.

10. A readable means used in a navigation system for a drilling operation in spinal surgery as in claim 9 wherein the comprising of the offering for the edit on the AP image, for the two-dimensional safety region for the left pedicle of the selected vertebra on an AP image, and for the two-dimensional safety region for the right pedicle of the selected vertebra on an AP image, and the edit on the Lat image for the two-dimensional safety region of the selected vertebra on an Lat image.

11. A readable means used in a navigation system for a drilling operation in spinal surgery as in claim 10 wherein the three-dimensional safety reconstruction for the selected vertebra comprises a three-dimensional safety volume of the left pedicle, which is the result of the projective intersection operation by the effective two-dimensional safety region of the left pedicle on an AP image and the effective two-dimensional safety region on a Lat image, and a three-dimensional safety volume of the right pedicle, which is the result of the projective intersection operation by the effective two-dimensional safety region of the right pedicle on an AP image and the effective two-dimensional safety region on a Lat image.

12. A readable means used in a navigation system for a drilling operation in spinal surgery as in claim 9 wherein the operation of inclination adjustment of the three-dimensional safety reconstruction is implemented by establishing an exit face and an enter face of the two-dimensional safety region on the AP image of the left pedicle and the AP image of the right pedicle respectively during planning the two-dimensional safety region, and followed by computing the horizontal distance between the enter face and the exit face, and establishing a two-dimensional safety region of the pedicle in profile on the Lat image, where the shape is like a parallelogram, and computing the maximum horizontal distance of the parallelogram as the height of the safety region of the pedicle, and the inclinations of the left and the right pedicles being then calculated to adjust the inclinations for the three-dimensional safety volume of the left and the right pedicles.
